(19) United States
(12) Patent Application Publication
(10) Pub. No.: US 2011/0165235 A1
(43) Pub. Date: Jul. 7, 2011

(54) DIRECTLY PRESSED ALISKIREN TABLETS

(75) Inventors: Jana Paetz, Moorenwies (DE); Frank Muskulus, Grobenzell (DE)

(73) Assignee: ratiopharm GmbH, Ulm (DE)

(21) Appl. No.: 13/003,612

(22) PCT Filed: Jul. 10, 2009
(86) PCT No.: PCT/EP2009/058801

§ 371 Date: Feb. 2, 2011

(30) Foreign Application Priority Data
Jul. 11, 2008 (EP) 08012558.6

(51) Int. Cl. A61K 31/165; C07C 237/22; A61K 31/549; A61K 9/28; A61P 9/12; A61P 9/04; A61P 9/00; A61P 27/06; A61P 25/00; A61P 25/22

(52) U.S. Cl. 424/465; 514/616; 564/157; 514/223.5

(57) ABSTRACT

The invention relates to pharmaceutical compositions which contain the active agent Aliskiren and are suitable for the production of tablets by dry pressing, so that prior wet granulation can be obviated. The invention also relates to tablets which can be obtained by dry pressing of these pharmaceutical compositions and to a method for producing these tablets. The invention furthermore relates to the use of the novel pharmaceutical compositions and tablets for treating hypertension and illnesses associated therewith.

The invention relates to pharmaceutical compositions which contain the active agent Aliskiren and are suitable for direct pressing, or dry processing, so that prior wet granulation of the active agent with auxiliaries can be obviated. The invention also relates to tablets which can be obtained by direct pressing of these pharmaceutical compositions and to a method for producing these tablets. The invention furthermore relates to the use of the novel pharmaceutical compositions and tablets for treating hypertension and illnesses associated therewith.

Aliskiren (IUPAC name: (2S,4S,5S,7S)-5-amino-N-(2-carbamoyl-2-methyl-propyl)-4-hydroxy-7-{[4-methoxy-3-(3-methoxypropoxy)phenyl]methyl}-8-methyl-2-propan-2-yl-nonanamide) is a direct renin inhibitor, which is used to reduce elevated blood pressure. Aliskiren has the following chemical structure:

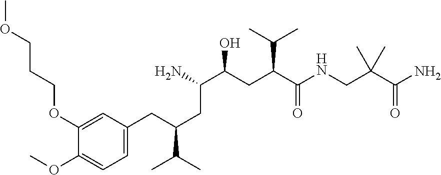

In particular, the hemifumarate salt of Aliskiren is known, as disclosed for example in EP 678 503 in Example 83. The hemifumarate has the following structure:

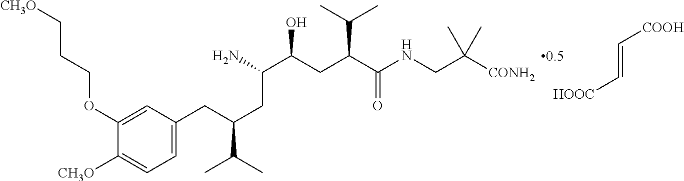

Unless otherwise indicated, the term “Aliskiren” in this application includes the free base as well as salts and solvates thereof.

In the prior art, administration forms of Aliskiren are known which have a high active agent content and release the active agent essentially without a time delay. WO 2004/002466 A1 describes the addition of surface-active media (“surfactants”) in order to increase the bioavailability of Aliskiren. Aliskiren is to be classed according to the “Biopharmaceutics Classification System” (BCS) as a class III active agent. This means that the active agent has a poor permeability but high solubility. The limiting factor for the bioavailability is therefore the permeation rate.

WO 2005/089729 A2 and US 2006/0018960 A1 disclose rapidly disintegrating Aliskiren tablets and Aliskiren tablets with immediate release, which are characterised by an active agent content >46%, expressed in terms of the total weight of the formulation. It is described that Aliskiren hemifumarate is difficult to process, inter alia since owing to its needle shape it is highly susceptible to interparticle bridge formation and, as a consequence of this, exhibits poor flowability and processability. In order to prevent problems concerning uniform distribution of the active agent in the medicament, it is therefore necessary to work with an active agent content >46%. Processing by wet granulation is furthermore described, which is likewise intended to counteract the poor properties of the active agent.

The two documents expressly disclose that direct pressing or dry compaction of the active agent is not possible owing to the high hygroscopicity, the needle-shaped particle structure and the poor flowability of the active agent. Correspondingly, WO 2005/089729 A2 and US 2006/0018960 A1 disclose a wet granulation method for producing medicaments which contain more than 46% Aliskiren. The multistage process of moist granulation, drying and subsequent pressing into tablets is in general relatively unproblematic in terms of its feasibility. Thus, generally, moist granulates can be tabletted most reliably and with the least complications, in particular even with low-dose administration forms. Moist granulation is therefore normally the method of choice, and moist granulates are the usual intermediate product in the production of tablets.

A disadvantage with moist granulation, however, is that special machines have to be used for the granulation, solvents are needed to produce the moist compound, and up to the end of drying the active agent is exposed to the granulating liquid for a prolonged period of time. This can lead to stability problems. Furthermore, the drying step which follows the granulation requires additional energy, so that the active agent is also exposed to thermal effects over a prolonged period of time. Overall, the moist granulation method is unsuitable in the case of a stability-labile active agent susceptible to polymorphism, in view of the moisture effect.

Document WO 2005/089729 A2 teaches that direct pressing of Aliskiren is not possible (page 2, last paragraph). As a solution, it is proposed to subject a high concentration of Aliskiren to wet granulation (Claim 1; Claim 19).

WO 2007/147596 A1 teaches that direct pressing of Aliskiren is not possible (page 3, first paragraph). As a solution, it is proposed to use a carbohydrate as a filler. Even with this, however, wet granulation is intended to be carried out (Claim 30).

WO 2008/074001 A1 relates to the use of Aliskiren for treating obese patients. This document does not deal with the difficult processability of Aliskiren; it merely refers to generally conventional production methods.

EP 1 972 335 A1 describes a method for producing medicaments containing Aliskiren, in which a granulate is produced first by melting. This method has the disadvantage that it is elaborate and the ingredients are exposed to higher temperatures, which can lead to partial breakdown of the active agent. The other disadvantages are the special requirements for the machines on an industrial scale, as well as the uneconomical high energy input in order to induce the melting.

There is therefore a need for pharmaceutical administration forms which contain the active agent Aliskiren and have advantages over the administration forms of the prior art. The pharmaceutical administration forms should in particular be producible simply and economically but nevertheless have good properties for administration. To this end, the active agent should be distributed as uniformly as possible inside the pharmaceutical administration form, even with an active agent content of less than 46 wt. %.

This object is achieved by the subject-matter of the claims. Surprisingly, it has been found that a pharmaceutical compensation which contains the active agent Aliskiren can be tabletted without prior moist granulation if the active agent has been mixed with a brittle auxiliary or a large amount of a lubricant and/or comminuted.

Unexpectedly, the tablets produced according to the invention are not susceptible to “capping” during the tabletting, even with high compression pressures. Capping refers to the splitting of one or more layers from the tablet surface during ejection from the die. In the case of active agents such as Aliskiren, which have a high plasticity and poor flowability, in dry pressing according to conventional methods a high susceptibility to capping is observed which is attributable to the high elastic recovery of plastic substances at the end of the pressing process.

Furthermore, with the method according to the invention it is possible to produce tablets which exhibit very rapid active agent release in the region of up to about 30 minutes. The tablets according to the invention are therefore outstandingly suitable as “immediate release” preparations. This effect is observed particularly in the case of formulations which are free from carbohydrates as fillers.

### SUMMARY OF THE INVENTION

The present invention relates in a first aspect to a pharmaceutical composition which is suitable for dry pressing into tablets, comprising the active agent Aliskiren or one of its pharmaceutically compatible solvates or salts and at least 10 wt. % of a brittle auxiliary, expressed in terms of the total weight of the pharmaceutical composition.

A further aspect of the invention is a pharmaceutical composition which is suitable for dry pressing into tablets, comprising the active agent Aliskiren or one of its pharmaceutically compatible solvates or salts and at least 5 wt. % of a lubricant, expressed in terms of the total weight of the pharmaceutical composition.

The invention relates according to a further aspect to a pharmaceutical composition which is suitable for direct pressing, or dry processing, into tablets, comprising the active agent Aliskiren or one of its pharmaceutically compatible solvates or salts, or a solvate of a salt, in free-flowing form. This means that Aliskiren is present with at least one other substance in a formulation which is free-flowing.

A further aspect of the invention is a pharmaceutical composition which is suitable for dry pressing into tablets, comprising the active agent Aliskiren or one of its pharmaceutically compatible solvates or salts, characterised in that the pharmaceutical composition is free from carbohydrates as fillers. Preferably, the pharmaceutical composition is entirely free from carbohydrates.

The invention likewise relates to the tablets which can be obtained from these pharmaceutical compositions by direct pressing, and to the method by which the compositions are pressed into tablets.

The invention relates in a further aspect to a method for producing a tablet, comprising direct pressing or dry compaction of a pharmaceutical composition of the present invention.

The invention furthermore relates to a method for producing a medicament, comprising the following steps:
(a) mixing crystalline or semicrystalline Aliskiren with 50-150 wt. % of a brittle auxiliary and/or with at least 10 wt. % of a lubricant, in each case expressed in terms of the total amount of Aliskiren in the medicament, and optionally with further auxiliaries;
(b) optionally compacting or dry granulating the mixture obtained in (a) and mixing the compacted Aliskiren with one or more auxiliaries;
(c) dry pressing the composition obtained in (a) or (b) into a tablet; and
(d) optionally coating the tablet obtained in this way.

A further aspect of the invention is a tablet which can be obtained by a method of the present invention.

A further aspect of the invention is a tablet containing Aliskiren, 20-70 wt. % of a brittle inorganic auxiliary, expressed in terms of the total weight of the tablet, and at least one disintegrant.

A further aspect of the invention is a tablet containing Aliskiren, at least 5 wt. % of a lubricant, expressed in terms of the total weight of the tablet, and at least one disintegrant.

A further aspect of the present invention is the use of a pharmaceutical composition according to the invention or a tablet according to the invention to produce a medicament for treating high blood pressure or an illness associated therewith.

### DETAILED DESCRIPTION OF THE INVENTION

Active Agent(s)

The pharmaceutical compositions according to the invention contain Aliskiren as active agent. All crystal forms of Aliskiren may be used, for example the crystal forms of Aliskiren hemifumarate described in WO 2008/061622 A1. The disclosure of WO 2008/061622 A1 is incorporated by reference into the disclosure of the present application.

The concentration of Aliskiren in the pharmaceutical composition according to the invention may be about 10 to 80 wt. %, expressed in terms of the total weight of the pharmaceutical composition. Preferably, the concentration of Aliskiren in the pharmaceutical composition according to the invention is about 10 to <46 wt. %, more preferably about 20 to 45 wt. %, most preferably about 35 to 42 wt. %, in each case expressed in terms of the total weight of the pharmaceutical composition. Unless otherwise indicated, the weight data of Aliskiren refer to the salt or solvate, not to the free base.

Besides Aliskiren, the compositions according to the invention may contain one or more further active agents selected from the group consisting of AT₁ receptor antagonists, ACE inhibitors, beta blockers, calcium channel blockers, aldosterone synthase inhibitors, aldosterone receptor antagonists and diuretics. Examples of these active agents are well known to the person skilled in the art. A preferred diuretic is hydrochlorothiazide (HCT). Most preferably, Aliskiren is combined with HCT. The HCT may be present with Aliskiren in a formulation, which is preferred. In another embodiment, Aliskiren and HCT are present in separate formulations, but in a medicinal combination. The concentration of HCT is normally in the range of from 5 to 50 mg per unit dosage form, preferably in the range of from 10 to 40 mg, more preferably in the range of from 12.5 to 30 mg, most preferably in the range of from 12.5 to 25 mg. The weight ratio of Aliskiren to HCT normally lies in the range of from 3:1 to 40:1, preferably from 5:1 to 30:1, more preferably from 7:1 to 25:1, most preferably from 10:1 to 20:1.

Aliskiren with Brittle Auxiliary

The first aspect of the invention is a pharmaceutical composition which is suitable for dry pressing into tablets, comprising the active agent Aliskiren or one of its pharmaceutically compatible solvates or salts and at least 10 wt. % of a brittle auxiliary, expressed in terms of the total weight of the pharmaceutical composition.

According to this aspect, the active agent Aliskiren is mixed with at least one brittle auxiliary.

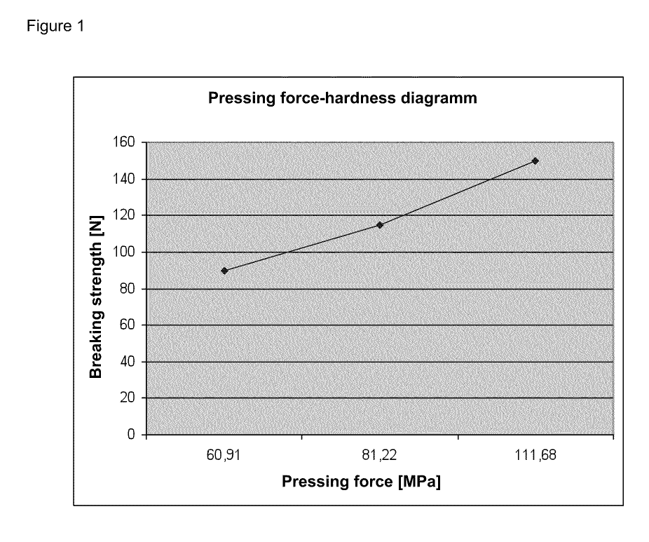

It has surprisingly been found that the pharmaceutical composition has a better pressability in the presence of a brittle auxiliary than in the absence of the brittle auxiliary. In this way, the formulation can readily be directly pressed into tablets, or processed by dry granulation. It has been possible to show that for various settings of the pressing force the hardness of the tablet increases, but without being susceptible to capping, as represented in a pressing force-hardness profile (see FIG. 1). With the aid of this graphical representation, a capping susceptibility of the tablet would be revealed by the formation of a plateau phase. Moist granulation is therefore not necessary.

Auxiliaries can generally be classified by means of their behaviour, or the change of the particle configuration under pressure (pressing): plastic auxiliaries are distinguished by plastic deformation, whereas brittle auxiliaries exhibit fracture of the particles into smaller particles when a pressing force is applied. A brittle behaviour of the auxiliary can be quantified by the increase of the surface area in the pressed article. In the literature, it is normal to classify the deformation mechanism with the aid of the so-called “yield pressure”. According to simple classification, the values of the yield pressure are small for plastic substances, whereas they are high for frangible substances [Duberg, M., Nyström, C., 1982. Studies on direct compression of tablets VI. Evaluation of methods for the estimation of particle fragmentation during compaction. Acta Pharm. Suec. 19, 421-436; Humbert-Droz P., Mordier D., Doelker E. Méthode rapide de détermination du comportement á la compression pour des études de préformulation {Rapid method for determining compression behaviour for preformulation studies}. Pharm. Acta Helv., 57, 136-143 (1982)]. The yield pressure describes the stress which must be reached so that the substance starts to flow plastically, and may be regarded as a measure of internal strength. It can be determined by the inverse value of the slope of the Heckel plot. Yield pressure values of less than 80 MPa are viewed as an indication of plastic flow behaviour (York, P., Drug Dev. Ind. Pharm. 18, 677 (1992); Crystal engineering and particle design for the powder compaction process). In the scope of the present invention, an auxiliary is taken to be a brittle auxiliary if it has a yield pressure of at least 80 MPa, determined according to the “ejected tablet” method, see W. A. Ritschel and A. Bauer-Brandl, “Die Tablette” {The tablet}, 2^{nd} edition, Editio Cantor Verlag Aulendorf, 2002, page 442 and the literature cited there.

The brittle auxiliary is preferably an inorganic brittle auxiliary. Inter alia, pharmaceutically acceptable inorganic salts of calcium or magnesium may be used as brittle auxiliaries, for example calcium carbonate, calcium phosphate, calcium sulphate, magnesium oxide. Calcium phosphate is preferred, in particular calcium hydrogen phosphate, and anhydrous calcium hydrogen phosphate is most preferred (available for example as Di-Cafos AN from Chemische Fabrik Budenheim).

The concentration of the brittle auxiliary in the pharmaceutical composition is generally about 10 to 80 wt. %, preferably 20-70 wt. %, more preferably 30-65 wt. %, most preferably 40-60 wt. %, for example about 50 wt. %, in each case expressed in terms of the total weight of the pharmaceutical composition.

The weight ratio of Aliskiren to brittle auxiliary is normally from 1:5 to 5:1, preferably 1:3 to 3:1, more preferably 1:2 to 2:1, most preferably 1:1 to 1:1.5.

Aliskiren with a Large Proportion of Lubricant

The second aspect of the invention is a pharmaceutical composition which is suitable for dry pressing into tablets, comprising the active agent Aliskiren or one of its pharmaceutically compatible solvates or salts and at least 5 wt. % of a brittle auxiliary, expressed in terms of the total weight of the pharmaceutical composition. This aspect of the invention may be combined with the other aspects described herein.

According to this aspect of the invention, the active agent Aliskiren is mixed with a relatively large proportion of at least one lubricant. It has been found that by mixing with a large proportion of lubricant, Aliskiren can be converted into a formulation which can readily be directly pressed into tablets, or processed by dry granulation.

All conventional pharmaceutical auxiliaries may be used as lubricants—preferably magnesium stearate or dehydrated plant oils (for example Lubritab®). Stearic acid, sodium stearyl fumarate and/or polyethylene glycol (PEG) may also be used as lubricants.

The amount of lubricant according to this aspect of the invention should be at least 5 wt. %, preferably from 5 to 30 wt. %, more preferably from 10 to 20 wt. %, in each case expressed in terms of the total weight of the pharmaceutical composition. For the other aspects of the invention the lubricant content may be lower, as indicated below.

Comminution of Aliskiren

In this embodiment of the invention, which may be combined with all the others, the active agent is processed by comminution technology before direct pressing or dry compaction. The crystal habit of the active agent is thereby modified, and the bridge formation is prevented. Since Aliskiren fumarate is susceptible to conversion under mechanical influence into the amorphous active agent, which has a lower stability, care is to be taken with the temperature during the comminution. The temperature should be kept below 40° C., preferably below 35° C., for example at about 10 to about 34° C. or about 20 to about 30° C. during the comminution, in order to prevent breakdown.

The active agent comminution is preferably carried out in the form of co-grinding with a surface-stabilising auxiliary. The addition of gum arabic as a surface-stabilising auxiliary during the co-grinding has been found to be suitable for this. The active agent becomes incorporated into the auxiliary during the co-grinding. Other possible surface stabilisers are mentioned below.

All known methods of pharmaceutical micronising or co-grinding may be employed, for example by using an air jet mill or ball mill. When using an air jet mill, it is preferable to work with process air at 4-6 bar. Processing under a protective gas atmosphere is likewise possible. The comminuted crystals preferably have an average size of 3-250 μm, preferably 5-150 μm, most preferably 10-120 μm.

The average particle size may be determined by laser diffraction according to Pharm. Eur. 2.9.31.Version 6.6, for example in a Malvern Mastersizer.

The micronising, or co-grinding, may be carried out with or without a surface stabiliser. Possible agglomeration of the micronised active agent may be counteracted by adding one or more surface stabilisers. These may be added before or during the comminution. Pharmaceutically compatible organic or inorganic auxiliaries which are polymers, low molecular weight oligomers or nonionic, ionic, zwitterionic surface-active substances, or contain them, may be used as surface stabilisers. They are added in an active agent/auxiliary weight ratio of from 1:1 to 1:100 preferably from 1:2 to 1:50. Examples of surface stabilisers in the context of the present invention are hydroxypropyl methyl cellulose (HPMC), casein, gelatines, polyvinyl pyrrolidone (PVP), sodium dodecyl sulphate (SDS), traganth, stearic acid, gum arabic, polaxomer, polyethylene glycol (PEG), Tween®, polysaccharides, alginates, phospholipids etc.

Coating of Aliskiren

In another embodiment, the crystal habit and the surface area of the crystals of the active agent may be modified by coating so that bridge formation is prevented and the flowability, and therefore the dosing accuracy, is increased. All conventional pharmaceutically compatible polymers may be used for this, for example cellulose-based polymers, methacrylates or polyvinyl alcohol (PVA). Examples of pharmaceutically compatible polymers are cellulose ethers such as hydroxypropyl cellulose, hydroxymethyl cellulose, methyl cellulose and hydroxypropyl methyl cellulose, cellulose acetate phthalate, hydroxypropyl methyl cellulose being particularly preferred. Suitable hydroxypropyl methyl celluloses are available for example under the brand name “Methocel”. References furthermore made to “Fiedler—Lexikon der Hilfsstoffe” {Fiedler—Lexicon of auxiliaries}, Editio Cantor-Verlag Aulendorf, 5^{th} edition 2002.

Owing to the good solubility properties of Aliskiren, this method also leads to smoothing by partial dissolving phenomena on the surface of the crystals, which is in turn advantageous for the further processing by direct pressing. The use of such polymers, which also constitute hydrophilising media, furthermore leads to improvement of the bioavailability of the active agent. The coating of the active agent is preferably carried out in a fluidised bed or by means of spray drying.

For the coating, the polymers are preferably used in an amount of 2-20 wt. %, more preferably 5-15 wt. %, most preferably 7-12 wt. %, in each case expressed in terms of the weight of the active agent. The coating solution is produced on the basis of an organic solvent. Suitable organic solvents are ethanol or isopropanol.

Depending on the polymer used, operation is normally carried out with a product temperature of between 30 and 45° C. or between 35 and 40° C.

A further advantage of this processing method is the stabilisation of the active agent against external influences, for example moisture, which promote conversion into the thermolabile amorphous state. As a result, the medicament can be put on the market in more economical packaging.

A variant of this technology is incorporation of the active agent into an auxiliary (preferably into a polymer) and heating of this solution on a carrier. To this end, the active agent is dissolved or suspended in a solution of a pharmaceutically compatible auxiliary (for example a polymer) and coated onto an inert core by known layering methods, for example a fluidised bed. Any type of “multiple units” are possible—pellets, minitablets, particles etc.

For a rapidly releasing formulation, it is preferable to use a mixture of a water-soluble auxiliary and a water-insoluble auxiliary. It is, however, also possible to use only water-insoluble or only water-soluble auxiliaries. Suitable water-soluble auxiliaries are for example HPMC, povidone, povidone VA 64 (copolymer of polyvinyl pyrrolidone with polyvinyl acetate, commercially available from BASF), mannitol, sorbitol. Suitable water-insoluble auxiliaries for example polyvinyl alcohol, cellulose acetate phthalate, various methacrylates etc.

This possibility combines both the improved stabilisation of the active agent by intimate embedding in a polymer, and the improved processability of the carrier-active agent mixture. As a result, no bridge formation of the active agent takes place, and reproducible production is ensured in respect of the uniform distribution of the active agent.

Aliskiren in a Formulation without Carbohydrates as Fillers

A further aspect of the invention, which is preferably combined with one or more of the aspects described further above, is a pharmaceutical composition which is suitable for dry pressing into tablets, comprising the active agent Aliskiren or one of its pharmaceutically compatible solvates or salts, characterised in that the pharmaceutical composition is free from carbohydrates as fillers. Preferably, the pharmaceutical composition is entirely free from carbohydrates.

The term “carbohydrates” in the context of the present invention comprises monosaccharides, disaccharides, oligosaccharides, polysaccharides and sugar alcohols. Modified carbohydrates are not carbohydrates in the context of this application. Modified carbohydrates are to be understood as chemical derivatives of carbohydrates, in particular not naturally occurring but artificially produced derivatives (for example croscarmellose, HPMC, HPC, HEC, CMC, carboxymethyl starch, methyl cellulose etc.).

In another embodiment, the pharmaceutical composition is free from carbohydrates as fillers and free from modified carbohydrates as fillers. In yet another embodiment, the pharmaceutical composition is entirely free from carbohydrates and entirely free from modified carbohydrates.

These embodiments also have the advantage that they do not contain lactose, and can therefore be taken by patients with lactose intolerance.

Properties of the Pharmaceutical Compositions

Contrary to what is stated in WO 2005/089729, the properties of Aliskiren in respect of flowability and processability can be improved by the method as described above, so that direct pressing or dry compaction is possible. Reproducible bulk and tamped densities can thereby be achieved, which ensure volume-dosed processing by tabletting and therefore “content uniformity”. It has also been found that, in particular, the addition of a brittle auxiliary can greatly improve this effect, or even exert a positive effect on the stability.

The combination of the auxiliaries should be selected so that the loss on drying of the tabletting mixture lies between 0.2-6%, preferably between 1.0-4%. The loss on drying is determined according to Pharm. Eur. 6.0-2.5.32. To this end, a temperature gradient is applied at 5°/min from 50-85° C.

Owing to the pretreatment of the active agent, the final mixtures exhibit good reproducibility in respect of the bulk and tamped densities and the ratio thereof. The bulk density preferably lies between 0.4-0.8 kg/l, and the ratio of tamped and bulk densities between 1.01 and 1.3 or between 1.1 and 1.3.

Pressing/Tabletting

The invention relates to a method for producing a medicament, comprising the following steps:
(a) mixing crystalline or semicrystalline Aliskiren with 50-150 wt. % of a brittle auxiliary or with at least 10 wt. % of a lubricant, in each case expressed in terms of the total amount of Aliskiren in the medicament, and optionally with further auxiliaries;
(b) optionally compacting or dry granulating the mixture obtained in (a) and mixing the compacted Aliskiren with one or more auxiliaries;
(c) dry pressing the composition obtained in (a) or (b) into a tablet; and
(d) optionally coating the tablet obtained in this way.

If step (b) is carried out, this is dry compaction or dry granulation. If step (b) is not carried out, this is direct pressing without granulation or precompaction.

A method for producing a medicament comprising the following steps is preferred:
(a) mixing crystalline or semicrystalline Aliskiren with 50-150 wt. % of a brittle auxiliary and/or with at least 10 wt. % of a lubricant, in each case expressed in terms of the total amount of Aliskiren in the medicament;
(b) directly pressing the composition obtained in (a) into a tablet; and
(c) optionally coating the tablet obtained in this way.

The pharmaceutical compositions of the present invention, produced by mixing and optionally compaction, can therefore be pressed into dried tablets. All conventional dry compaction methods by roller compaction or briquette compaction may be used for this. Care should be taken that the pressing force lies between 2-50 kN, preferably between 3 and 30 kN, for example 5-20 kN.

The active agent can thus be pressed with conventional pharmaceutical auxiliaries into a tablet, which may then also be coated in order to mask the taste of the bitter active agent.

Unless otherwise indicated, the following auxiliaries may be used:

As a binder, it is preferable to use a water-soluble auxiliary, for example povidone, HPMC, HPC. As a water-soluble auxiliary, any pharmaceutically compatible material with a neutral taste may be used. The amount of binder may be about 10 to 25 wt. %, preferably 15 to 25 wt. %, most preferably 17 to 24 wt. %, in each case expressed in terms of the total weight of the pharmaceutical composition.

A filler or diluent may be added: for example lactose and/or other sugar alcohols or carbohydrates. These may be combined with a proportion of water-insoluble fillers, preferably with microcrystalline cellulose. Other inorganic salts may, however, also be envisaged as fillers: calcium carbonate, calcium hydrogen phosphate etc. The amount of filler may be about 10 to 80 wt. %, preferably 10 to 70 wt. %, most preferably 15 to 60 wt. %, in each case expressed in terms of the total weight of the pharmaceutical composition.

In order to ensure rapid breakdown and the release correlated therewith, a disintegrant should be used: preferably crospovidone, sodium carboxymethyl starch or similar superdisintegrants. The amount of disintegrant is generally about 1 to 20 wt. %, preferably 2 to 10 wt. %, most preferably 3 to 8 wt. %, in each case expressed in terms of the total weight of the pharmaceutical composition.

All conventional pharmaceutical auxiliaries may be used as a lubricant—preferably magnesium stearate or dehydrated plant oils (for example Lubritab®). Stearic acid, sodium stearyl fumarate and/or polyethylene glycol (PEG) may also be used as a lubricant. The amount of lubricant may be about 1 to 10 wt. %, preferably 2 to 8 wt. %, most preferably 3 to 7 wt. %, in each case expressed in terms of the total weight of the pharmaceutical composition.

The constituents are conventionally mixed together in a freefall mixer (Turbula) and subsequently pressed into tablets on a rotary press (Fette).

In this case, the tabletting mixtures exhibited good compressibility. The main compression forces applied normally lie in the range of from 2 to 50 kN, preferably from 5 to 12 kN, depending on the size and type of the press being used.

The present invention furthermore relates to a method for producing a medicament, comprising the following steps:
comminuting Aliskiren crystals or semicrystalline Aliskiren with or without a surface stabiliser;
optionally mixing the comminuted Aliskiren crystals with one or more auxiliaries;
directly pressing the composition obtained in this way, in order to obtain a tablet; and
optionally coating the tablet obtained in this way.

The invention furthermore relates to a method for producing a medicament, comprising the following steps:
coating crystalline Aliskiren or semicrystalline Aliskiren with at least one pharmaceutically compatible polymer;
optionally mixing the coated Aliskiren with one or more auxiliaries;
directly pressing the composition obtained in this way into a tablet; and
optionally coating the tablet obtained in this way.

The invention furthermore relates to a method for producing a medicament, comprising the following steps:
compacting crystalline Aliskiren or semicrystalline Aliskiren with at least one pharmaceutically compatible polymer;
optionally mixing the compacted Aliskiren with one or more auxiliaries;
directly pressing the composition obtained in this way into a tablet; and

optionally coating the tablet obtained in this way.

In preferred embodiments of the method according to the invention, no wet granulation or moist granulation is carried out, in particular no aqueous wet or moist granulation.

If dry granulation is carried out, the temperature during the granulation is preferably below the melting point of all the auxiliaries used. For example, the temperature is lower than 40° C., or lower than 35° C. So-called “hot-melt” granulation is therefore not preferred.

Properties of the Tablets

The tablets according to the invention generally have a hardness of between 60-160 N, preferably 60-120 N, more preferably 70-110 N. The hardness may be determined by methods known per se, for example Pharm. Eur. 6.0 <2.9.8>.

The tablets according to the invention exhibit low rolling and shaking wear (friability), i.e. good abrasion strength. The friability is preferably less than 1% (for example 0.1 to 0.8%, preferably 0.2 to 0.5%). The friability may be determined by methods known per se, for example according to Pharm. Eur. 4.0/2.09.07.00.

The tablets according to the invention generally have a geometry which is adapted to the amount of active agent contained.

A particular advantage of the tablets according to the invention is their advantageous release profile. The tablets release a substantial proportion of the active agent within 30 minutes, measured according to USP 28, Method <711>, Apparatus 2, in 500 ml 0.1 HCl pH 1.1 at 37° C. and 75 rpm. In a first embodiment, the tablets exhibit active agent release of at least 15% after 5 minutes, at least 40% after 10 minutes, at least 60% after 15 minutes, and at least 70% after 20 minutes, determined according to USP 28, Method <711>, Apparatus 2, in 500 ml 0.1 HCl pH 1.1 at 37° C. and 75 rpm.

In a second embodiment, the tablets exhibit active agent release of at least 20% after 5 minutes, at least 50% after 10 minutes, at least 70% after 15 minutes, and at least 80% after 20 minutes, determined according to USP 28, Method <711>, Apparatus 2, in 500 ml 0.1 HCl pH 1.1 at 37° C. and 75 rpm.

In a third embodiment, the tablets exhibit active agent release of at least 20% after 5 minutes, at least 50% after 10 minutes, at least 80% after 15 minutes, and at least 90% after 20 minutes, determined according to USP 28, Method <711>, Apparatus 2, in 500 ml 0.1 HCl pH 1.1 at 37° C. and 75 rpm.

In a fourth embodiment, the tablets exhibit active agent release after 5 minutes of at least 10%, preferably at least 15%, most preferably at least 20%, determined according to USP 28, Method <711>, Apparatus 2, in 500 ml 0.1 HCl pH 1.1 at 37° C. and 75 rpm.

In a fifth embodiment, the tablets exhibit active agent release after 10 minutes of at least 30%, preferably at least 40%, more preferably at least 50%, most preferably at least 60%, or even at least 75% determined according to USP 28, Method <711>, Apparatus 2, in 500 ml 0.1 HCl pH 1.1 at 37° C. and 75 rpm.

In a sixth embodiment, the tablets exhibit active agent release after 15 minutes of at least 50%, preferably at least 60%, more preferably at least 70%, most preferably at least 80%, or even at least 90% determined according to USP 28, Method <711>, Apparatus 2, in 500 ml 0.1 HCl pH 1.1 at 37° C. and 75 rpm.

In a seventh embodiment, the tablets exhibit active agent release after 20 minutes of at least 70%, preferably at least 75%, more preferably at least 80%, most preferably at least 90%, determined according to USP 28, Method <711>, Apparatus 2, in 500 ml 0.1 HCl pH 1.1 at 37° C. and 75 rpm.

After 30 minutes, the tablets normally exhibit an active agent release of at least 90%, usually at least 95%, determined according to USP 28, Method <711>, Apparatus 2, in 500 ml 0.1 HCl pH 1.1 at 37° C. and 75 rpm.

In a further embodiment, the tablets exhibit a release profile which is similar to that of the medicament Rasilez®, or which is essentially the same as the release profile of Rasilez®. By formulation additives which are known per se, the person skilled in the art can delay the very rapid active agent release of the tablets described above, so as to obtain a release profile which is the same as that of Rasilez® or is similar to it (in each case at most 10% difference in the active agent release after 5, 10, 15, 20 and 30 minutes).

Uses

The invention furthermore relates to the use of the pharmaceutical composition or tablet described above for treating high blood pressure or an illness associated therewith. These illnesses include inter alia congestive heart failure, cardiohypertension, cardiofibrosis, postinfarct cardiomyopathy, complications as a consequence of diabetes, such as nephropathy, vasculopathy and neuropathy, coronary vascular diseases, restenosis after an angioplasty, increased intraocular pressure, glaucoma, abnormal vascular growth, hyperaldosteronism, states of anxiety and cognition disorders.

The invention furthermore relates to the use of Aliskiren to produce a medicament for treating high blood pressure or an illness associated therewith, characterised in that a pharmaceutical composition containing Aliskiren is directly pressed into tablets. The active agent Aliskiren is preferably coated with at least one pharmaceutically compatible polymer before the direct pressing. As an alternative, the Aliskiren crystals or semicrystalline Aliskiren may be comminuted before the direct pressing, for example by micronising as described above.

According to the invention, it is also possible for the pharmaceutical compositions and tablets described herein to be administered in combination therapy. In this case, the medicament according to the invention may be administered simultaneously with a further active agent or at different times. For example, the following active agent categories may be envisaged as further active agents: AT, receptor antagonists, ACE inhibitors, beta blockers, calcium channel blockers, aldosterone synthase inhibitors, aldosterone receptor antagonists and diuretics. Examples of these active agents are well known to the person skilled in the art. A preferred diuretic is hydrochlorothiazide (HCT).

Most preferably, Aliskiren is combined with HCT. The HCT may be present with Aliskiren in a formulation, which is preferred. In another embodiment, Aliskiren and HCT are present in separate formulations, but in a medicinal combination. The concentration of HCT is normally in the range of from 5 to 50 mg per unit dosage form, preferably in the range of from 10 to 40 mg, more preferably in the range of from 12.5 to 35 mg, most preferably in the range of from 12.5 to 25 mg.

The weight ratio of Aliskiren to HCT normally lies in the range of from 3:1 to 40:1, preferably from 5:1 to 30:1, more preferably from 7:1 to 25:1, most preferably from 10:1 to 20:1.

The present invention furthermore relates to the following subjects (1) to (16):

(1) Pharmaceutical composition which is suitable for the direct pressing of tablets, comprising the active agent Aliskiren or one of its pharmaceutically compatible solvates or salts in free-flowing form.

(2) Pharmaceutical composition according to (1), characterised in that the active agent is present in micronised form.

(3) Pharmaceutical composition according to (1) or (2), characterised in that the active agent is coated with a pharmaceutically compatible polymer.

(4) Pharmaceutical composition according to (3), characterised in that the pharmaceutically compatible polymer is selected from the group consisting of cellulose-based polymers, methacrylates and polyvinyl alcohol.

(5) Pharmaceutical composition according to (3) or (4), characterised in that the content of the pharmaceutically compatible polymer is 2-20 wt. %, expressed in terms of the weight of the active agent.

(6) Pharmaceutical composition according to one of the preceding subject-matters (1) to (5), characterised in that it contains a binder.

(7) Pharmaceutical composition according to one of the preceding subject-matters (1) to (6), characterised in that the bulk density of the composition lies between 0.4 and 0.7 kg/l.

(8) Pharmaceutical composition according to one of the preceding subject-matters (1) to (7), characterised in that the ratio of tamped density and bulk density lies between 1.1 and 1.3.

(9) Method for producing a tablet, comprising the direct pressing of a pharmaceutical composition according to one of the preceding subject-matters (1) to (8).

(10) Method according to (9), characterised in that the pressing force during the direct pressing lies between 2 and 50 kN.

(11) Tablet which can be obtained by a method according to (9) or (10).

(12) Tablet containing micronised Aliskiren or Aliskiren coated with a pharmaceutically compatible polymer.

(13) Use of a pharmaceutical composition according to one of subject-matters (1) to (8) or a tablet according to (11) or (12) to produce a medicament for treating high blood pressure or an illness associated therewith.

(14) Use according to (13), characterised in that the illness associated with high blood pressure is selected from the group consisting of congestive heart failure, cardiohypertension, cardiofibrosis, postinfarct cardiomyopathy, complications as a consequence of diabetes, such as nephropathy, vasculopathy and neuropathy, coronary vascular diseases, restenosis after an angioplasty, increased intraocular pressure, glaucoma, abnormal vascular growth, hyperaldosteronism, states of anxiety and cognition disorders.

(15) Use according to (13) or (14), characterised in that the pharmaceutical composition or tablet is administered in combination with a further active agent.

(16) Use according to (15), characterised in that the further active agent is selected from the group consisting of AT, receptor antagonists, ACE inhibitors, beta blockers, calcium channel blockers, aldosterone synthase inhibitors, aldosterone receptor antagonists and diuretics.

The above subject-matters (1) to (16) may furthermore be combined with the aspects and embodiments described in the present application.

FIG. 1 shows a pressing force-hardness diagram with the results of Example 8.

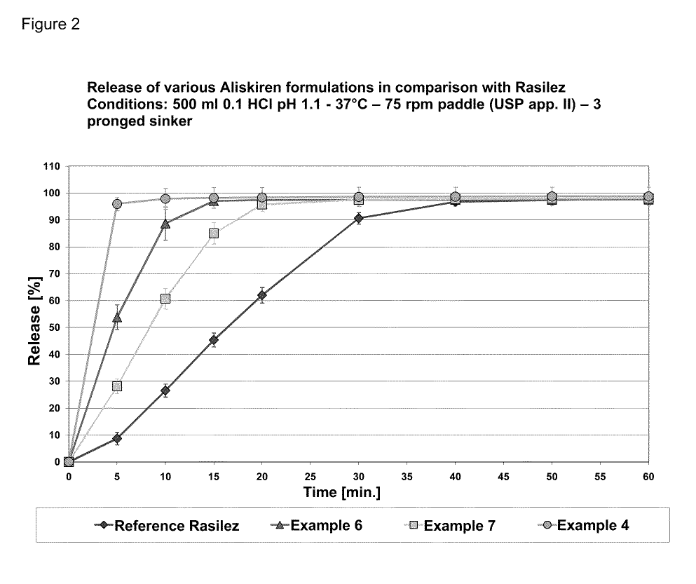

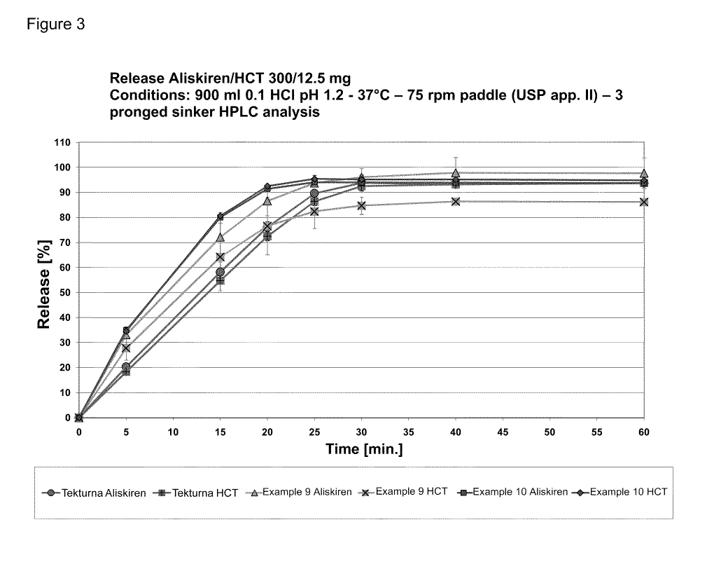

FIGS. 2 and 3 shows the release profiles which were found in Examples 11a and 11b, respectively.

The invention will be further illustrated by the following examples:

### EXAMPLE 1

### Dry Compaction with Calcium Phosphate

Quantity data are used as calculated for the individual dose.

The active agent was compacted with 300 mg of calcium phosphate, 5 mg of Aerosil, 40 mg of croscarmellose and 20 mg of magnesium stearate in a compactor at 15-45 kN. The compactate was comminuted using a screen and mixed with the remaining amounts of calcium phosphate, magnesium stearate and Aerosil on a freefall mixer. This final mixture was pressed into tablets on a rotary press. They had a hardness of 70-110 N combined with a friability of less than 1%. The tablets were subsequently coated in a drum coater. A suspension of HPMC, PEG, talc, titanium dioxide and the colorant were used for this.

TABLE 1

| | | | | | % per |
|---|---|---|---|---|---|
| No. | Ingredient | Product | Function | [mg] | D.F. |
| 1 | Aliskiren | Hemifumarate | active agent | 331.5* | 37.7 |
| 2 | Calcium | Di-Cafos AN | diluent | 412.5 | 46.9 |
| | phosphate | | | | |
| 3 | Colloidal silicon | Aerosil | lubricant | 10.0 | 1.1 |
| | dioxide | | | | |
| 4 | Croscarmellose | Ac-Di Sol | disintegrant | 40.0 | 4.5 |
| | sodium | | | | |
| 5 | Magnesium | Stearate Mg | lubricant | 45.0 | 5.1 |
| | stearate | | | | |
| 6 | Hydroxypropyl | Methocel E5 | film former | 30.0 | 3.4 |
| | methyl cellulose | premium LV | | | |
| 7 | Polyethylene | Polyglycol | plasticiser | 4.5 | 0.5 |
| | glycol | 8000P | | | |
| 8 | Talc | Talc, micron. | antisticking | 3.5 | 0.4 |
| | | | agent | | |
| 9 | Titanium dioxide | Titanium | colorant | 2.5 | 0.3 |
| | | dioxide | | | |
| 10 | Colorant | Not specified | colorant | 0.5 | 0.1 |
| | Total | | | 880.0 | 100.0 |

*corresponding to 300 mg of Aliskiren base

### EXAMPLE 2

### Active Agent Coating

The active agent was placed in a fluidised bed device (Glatt GPC3) and coated with an isopropanolic solution of HPMC. The product temperature was between 35-40° C. with an applied air temperature of 40-80 ° C. The spraying pressure was set at 1-2 bar. The coated active agent was subsequently premixed with Avicel, Aerosil and croscarmellose in a freefall mixer. The end mixture was produced with the addition of magnesium stearate. For this purpose, mixing was carried out for a further 3 min. The final mixture was subsequently pressed on a rotary press (Fette 102i) into tablets with a hardness of 60-120 N.

If necessary, the tablets may be coated in an equivalent way to Example 1.

TABLE 2

| | | | | | % per |
|---|---|---|---|---|---|
| No. | Ingredient | Product | Function | [mg] | D.F. |
| 1 | Aliskiren | Hemifumarate | active agent | 331.5* | 39.0 |
| 2 | Hydroxypropyl | Methocel E5 | coating film | 30.0 | 3.5 |
| | methyl cellulose | | former | | |
| 3 | Microcrystalline | Avicel | diluent | 436.0 | 51.3 |
| | cellulose | | | | |
| 4 | Colloidal silicon | Aerosil | glidant | 2.5 | 0.3 |
| | dioxide | | | | |
| 5 | Croscarmellose | Ac-Di Sol | disintegrant | 35.0 | 4.1 |
| | sodium | | | | |
| 6 | Magnesium | Stearate Mg | lubricant | 15.0 | 1.8 |
| | stearate | | | | |
| | Total | | | 850.0 | 100.0 |

*corresponding to 300 mg of Aliskiren base

### EXAMPLE 3

### Active Agent Comminution (Co-Grinding with Gum Arabic)

The active agent was comminuted together with gum arabic in an air jet mill. Process air was applied at between 4-6 bar. After the grinding, the active agent-auxiliary mixture was mixed with Aerosil, crospovidone and Avicel for 30 min on a freefall mixer. Stirring was subsequently carried out for a further 3 min with magnesium stearate. The final mixture was pressed and optionally coated in a similar way to the previous examples.

TABLE 3

| | | | | | % per |
|---|---|---|---|---|---|
| No. | Ingredient | Product | Function | [mg] | D.F. |
| 1 | Aliskiren | Hemifumarate | active agent | 331.5* | 37.6 |
| 2 | Gum arabic | Gum arabic | surface | 331.5 | 37.6 |
| | | | stabiliser | | |
| 3 | Microcrystalline | Avicel | diluent | 120.0 | 13.7 |
| | cellulose | | | | |
| 4 | Colloidal silicon | Aerosil | glidant | 8.0 | 0.9 |
| | dioxide | | | | |
| 5 | Crosslinked | Crospovidone | disintegrant | 80.0 | 9.0 |
| | povidone | | | | |
| 6 | Magnesium | Stearate Mg | lubricant | 10.0 | 1.2 |
| | stearate | | | | |
| 7 | Total | | | 881.0 | 100.0 |

*corresponding to 300 mg of Aliskiren base

### EXAMPLE 4

### Direct Pressing with Calcium Phosphate

Calcium hydrogen phosphate, Aerosil 200, Ac-Di-Sol and magnesium stearate were screened through a 0.5 mm screen into the mixing container. Aliskiren hemifumarate was added. Mixing was subsequently carried out for 15 min on a Turbula T10B mixer. The final mixture obtained in this way was pressed by means of a hydraulic one-hand press (Specac hydraulic press) with a setting of 0.5 kN.

TABLE 4

| No. | Ingredient | Product | Function | [mg] |
|---|---|---|---|---|
| 1 | Aliskiren | Hemifumarate | active agent | 165.75* |
| 2 | Calcium phosphate | Di-Cafos AN | diluent | 206.25 |
| 3 | Colloidal silicon | Aerosil 200 | glidant | 5.00 |
| | dioxide | | | |
| 4 | Croscarmellose | Ac-Di Sol | disintegrant | 20.00 |
| | sodium | | | |
| 5 | Magnesium | Stearate Mg | lubricant | 22.50 |
| | stearate | | | |
| | Total | | | 419.50 |

*corresponding to 150 mg of Aliskiren base

The final mixture exhibited good pressability. The tablets had a sufficient hardness.

### EXAMPLE 5

### Direct Pressing without Carbohydrate

Calcium hydrogen phosphate, Aerosil 200, Kollidon CL and magnesium stearate were screened through a 0.5 mm screen into the mixing container. Aliskiren hemifumarate was added. Mixing was subsequently carried out for 15 min on a Turbula T10B mixer. The final mixture obtained in this way was pressed by means of a hydraulic one-hand press (Specac hydraulic press) with a setting of 0.5 kN.

TABLE 5

| No. | Ingredient | Product | Function | [mg] |
|---|---|---|---|---|
| 1 | Aliskiren | Hemifumarate | active agent | 165.75* |
| 2 | Calcium phosphate | Di-Cafos AN | diluent | 206.25 |
| 3 | Colloidal silicon | Aerosil 200 | glidant | 5.00 |
| | dioxide | | | |
| 4 | Crosslinked | Kollidon CL | disintegrant | 20.00 |
| | polyvinyl | | | |
| | pyrrolidone | | | |
| 5 | Magnesium | Stearate Mg | lubricant | 22.50 |
| | stearate | | | |
| | Total | | | 419.50 |

*corresponding to 150 mg of Aliskiren base

The final mixture exhibited good pressability. The tablets had a sufficient hardness.

### EXAMPLE 6

### Dry Compaction (Dry Granulation) with Calcium Phosphate

150 mg of calcium hydrogen phosphate, 2.5 mg of Aerosil 200, 20 mg of croscarmellose sodium and 10 mg of magnesium stearate were screened through a 0.5 mm screen into the mixing container. Aliskiren hemifumarate was added. Mixing was subsequently carried out for 15 min on a mixer (Turbula T10B). The mixture was pressed at about 0.1 kN on a hydraulic one-hand press (Specac). The compactate was subsequently comminuted through a 1 mm screen. The remaining amounts of calcium hydrogen phosphate and Aerosil were added through a 0.5 mm screen and mixed for a further 15 min. The remaining amounts of magnesium stearate were also added through a 0.5 mm screen, and the whole mixture was mixed on a Turbula T10B mixer for 5 min. The final mixture obtained in this way was pressed by means of a hydraulic one-hand press (Specac hydraulic press) with a setting of 10 kN.

TABLE 6

| No. | Ingredient | Product | Function | [mg] |
|---|---|---|---|---|
| 1 | Aliskiren | Hemifumarate | active agent | 165.75* |
| 2 | Calcium phosphate | Di-Cafos AN | diluent | 206.25 |
| 3 | Colloidal silicon | Aerosil 200 | glidant | 5.00 |
| | dioxide | | | |
| 4 | Croscarmellose | Ac-Di Sol | disintegrant | 20.00 |
| | sodium | | | |
| 5 | Magnesium | Stearate Mg | lubricant | 22.50 |
| | stearate | | | |
| | Total | | | 419.50 |

The final mixture exhibited good pressability. The tablets had a sufficient hardness.

### EXAMPLE 7

### Active Agent Comminution (Co-Grinding with Gum Arabic)

Aliskiren hemifumarate and gum arabic were mixed for 5 min in a Turbula T10B, followed by comminution in a mortar. Monocrystalline cellulose, Aerosil and Colidon CL were added through a 0.5 mm screen and mixed for 15 min. Magnesium stearate was added through a 0.5 mm screen, and the mixture was mixed for a further 3 min. The final mixture was pressed at 6-8 kN using a hydraulic one-hand press (Specac).

TABLE 7

| No. | Ingredient | Product | Function | [mg] |
|---|---|---|---|---|
| 1 | Aliskiren | Hemifumarate | active agent | 165.75* |
| 2 | Colloidal silicon | Aerosil 200 | glidant | 4.00 |
| | dioxide | | | |
| 3 | Magnesium | Stearate Mg | lubricant | 5.00 |
| | stearate | | | |
| 4 | Gum arabic | Gum arabic | surface stabiliser | 165.75 |
| 5 | Crosslinked | Kollidon XL | disintegrant | 40.00 |
| | polyvinyl | | | |
| | pyrrolidone | | | |
| 6 | Microcrystalline | Avicel PH 102 | diluent | 60.00 |
| | cellulose | | | |
| | Total | | | 440.50 |

The final mixture exhibited good pressability. The tablets had a sufficient hardness.

### EXAMPLE 8

### Direct Pressing with Different Compression Forces

Three formulations A, B and C with the composition represented in Table 7 were produced. Production was carried out as described in Example 4, with the difference that a “Riva piccola Rotary Press” having the parameters specified in Table 8 was used for the pressing

The composition of the formulations tested is given below:

TABLE 8

| Constituent | [mg/tablet] | [%] |
|---|---|---|
| Aliskiren hemifumarate | 169.24 | 40.01 |
| Di-Cafos AN | 206.25 | 48.76 |
| Aerosil 200 | 5.00 | 1.18 |
| Ac-Di-Sol | 20.00 | 4.73 |
| Magnesium stearate | 22.50 | 5.32 |
| Total | 422.99 | 100.00 |

The compression parameters for the three formulations are given below in Table 9.

TABLE 9
Compression Parameters and Measured Tablet Properties

| | Batch No | | |
|---|---|---|---|
| | A | B | C |
| Main compression force [kN] | 6.00 | 8.00 | 11.00 |
| Compression pressure [MPa] | 60.91 | 81.22 | 111.68 |
| Precompression force [kN] | 0.50 | 0.50 | 0.50 |
| Rotation speed [rpm] | 10 | 10 | 10 |
| Weight [mg] | 423.00 | 423.00 | 423.00 |
| Hardness [N] | 90.00 | 115.00 | 150.00 |
| Diameter [mm] | 11.00 | 11.00 | 11.00 |
| Thickness [mm] | 5.00 | 4.70 | 4.50 |

The compression pressure and the hardness are compared in FIG. 1 in a diagram. The curve exhibits no plateau at high pressures, which shows that the tablets are not susceptible to capping.

### EXAMPLE 9

### Combination with HCT—Direct Pressing with Calcium Phosphate

Calcium hydrogen phosphate, Aerosil 200, calcium phosphate and magnesium stearate were screened through a 0.5 mm screen into the mixing container. Aliskiren hemifumarate and HCT were added. Mixing was subsequently carried out for 15 min on a Turbula T10B mixer. The final mixture obtained in this way was pressed at about 0.1 kN by means of a hydraulic one-hand press (Specac hydraulic press) with a setting of 0.6-0.8 kN.

TABLE 10

| No. | Ingredient | Product | Function | [mg] |
|---|---|---|---|---|
| 1 | Aliskiren | Hemifumarate | active agent | 338.48* |
| 2 | HCT | | active agent | 12.50 |
| 3 | Calcium phosphate | Di-Cafos AN | diluent | 412.50 |
| 4 | Colloidal silicon | Aerosil 200 | glidant | 10.00 |
| | dioxide | | | |
| 5 | Crosslinked | Kollidon CL | disintegrant | 40.00 |
| | povidone | | | |
| 6 | Magnesium | Stearate Mg | lubricant | 45.00 |
| | stearate | | | |
| | Total | | | 858.48 |

### EXAMPLE 10

### Combination with HCT—Co-Grinding with PVP

Aliskiren hemifumarate, HCT and povidone were mixed for 5 min in a Turbula T10B, followed by comminution in a mortar and subsequently screening over 0.5 mm. Microcrystalline cellulose, Aerosil and Kollidon CL were added through a 0.5 mm screen and mixed for 15 min. Magnesium stearate was added through a 0.5 mm screen, and the mixture was mixed for a further 3 min. The final mixture was pressed at 6-8 kN using a hydraulic one-hand press (Specac).

TABLE 11

| No. | Ingredient | Product | Function | [mg] |
|---|---|---|---|---|
| 1 | Aliskiren | Hemifumarate | active agent | 338.48* |
| 2 | HCT | | active agent | 12.50 |
| 3 | Polyvinyl | Povidone 12.5 | surface | 160.00 |
| | pyrrolidone | | stabiliser | |
| 4 | Microcrystalline | Avicel PH 102 | filler | 120.00 |
| | cellulose | | | |
| 5 | Colloidal silicon | Aerosil 200 | glidant | 8.00 |
| | dioxide | | | |
| 6 | Crosslinked | Kollidon CL | disintegrant | 40.00 |
| | povidone | | | |
| 7 | Magnesium | Stearate Mg | lubricant | 10.00 |
| | stearate | | | |
| | Total | | | 688.98 |

### EXAMPLE 11

### Release Profile

a) The active agent release of the formulations according to Examples 4, 6 and 7 was determined according to USP 28, <711>, Apparatus 2 (Paddle). The commercially available formulation which is marketed under the name “Rasilez® 150 mg” was also tested as a reference product. The tablets produced in Examples 4, 6 and 7, and the reference product, were each stirred in 500 ml 0.1 N HCl, pH 1.1 at 37° C. with 75 rpm. The amount of active agent which had entered into solution was determined by means of HPLC. The results are summarised in FIG. 2.

As can be seen from FIG. 2, the formulations according to the invention exhibit a much more rapid active agent release than the reference product, in particular for the period up to 30 min.

b) The active agent release of the formulations of Examples 9 and 10 was determined according to USP 28, <711>, Apparatus 2 (Paddle). The commercially available formulation which is marketed under the name “Tekturna® 150 mg” was also tested as a reference product. The tablets produced in Examples 9 and 10, and the reference product, were each stirred in 900 ml 0.1 N HCl, pH 1.2 at 37° C. with 75 rpm. The amounts of both active agents which had entered into solution were determined by means of HPLC. The results are summarised in FIG. 3.

### EXAMPLE 12

### Loss on Drying

The loss on drying of the tablets produced according to Examples 4 and 8 was determined according to Pharm. Eur. 2.2.32. The results are given in Table 12 below.

TABLE 12

| | Example 4 | Example 9 |
|---|---|---|
| Loss on drying [%] | 1.7 | 3.4 |

Abbreviations

CMC Carboxymethyl cellulose

HEC Hydroxyethyl cellulose

HPC Hydroxypropyl cellulose

HPLC “High Performance Liquid Chromatography”

HPMC Hydroxypropyl methyl cellulose

IR “Immediate Release”

Pharm. Eur. European Pharmacopoeia

rpm revolutions per minute

USP United States Pharmacopeia

1. A pharmaceutical composition suitable for dry pressing into tablets comprising the active agent Aliskiren or one of its pharmaceutically compatible solvates or salts and at least 10 wt. % of a brittle auxiliary, expressed in terms of the total weight of the pharmaceutical composition.

2. A pharmaceutical composition suitable for dry pressing into tablets comprising the active agent Aliskiren or one of its pharmaceutically compatible solvates or salts and at least 5 wt. % of a lubricant, expressed in terms of the total weight of the pharmaceutical composition.

3. A pharmaceutical composition suitable for the direct pressing of tablets comprising the active agent Aliskiren or one of its pharmaceutically compatible solvates or salts in free-flowing form.

4. The pharmaceutical composition according to claim 1, wherein the active agent is present in the composition in micronised form.

5. The pharmaceutical composition according to claim 2, wherein said composition further comprises a brittle auxiliary.

6. The pharmaceutical composition according to claim 1, wherein the brittle auxiliary is selected from the group consisting of pharmaceutically compatible inorganic salts of calcium and pharmaceutically compatible inorganic salts of magnesium.

7. The pharmaceutical composition according to claim 6, wherein the brittle auxiliary is selected from the group consisting of calcium carbonate, calcium phosphate, calcium sulphate and magnesium oxide.

8. The pharmaceutical composition according to claim 7, wherein the brittle auxiliary is calcium hydrogen phosphate.

9. The pharmaceutical composition according to claim 1, wherein the brittle auxiliary content is ranges from 30 to 70 wt. %, expressed in terms of the total weight of the pharmaceutical composition.

10. The pharmaceutical composition according to claim 1, wherein said composition is free from carbohydrates fillers.

11. The pharmaceutical composition according to claim 1, wherein said composition is free from carbohydrates.

12. The pharmaceutical composition according to claim 1, further wherein the Aliskiren content, expressed in terms of the total weight of the pharmaceutical composition, is less than 46 wt. %.

13. The pharmaceutical composition according to claim 1, wherein said composition further comprises hydrochlorothiazide as an additional active agent.

14. The pharmaceutical composition according to claim 1, wherein the bulk density of the composition ranges between 0.4 and 0.8 kg/l.

15. The pharmaceutical composition according to claim 1, wherein the ratio of tamped density to bulk density ranges from 1.01 to 1.3.

16. The pharmaceutical composition according to claim 1, wherein said composition contains at least one further active agent selected from the group consisting of AT₁ receptor antagonists, ACE inhibitors, beta blockers, calcium channel blockers, aldosterone synthase inhibitors, aldosterone receptor antagonists and diuretics.

17. The pharmaceutical composition according to claim 16, wherein the further active agent is hydrochlorothiazide.

18. A method for producing a tablet form of Aliskiren, said method comprising the step of direct pressing the pharmaceutical composition according to claim 1.

19. The method according to claim 18, wherein the step of direct pressing is characterised by a pressing force that ranges between 2 and 50 kN.

20. A method for producing a tablet form of Aliskiren, said method comprising the step of dry compacting the pharmaceutical composition according to claim 1.

21. A method for producing a tablet form of Aliskiren, said method comprising the following steps:
(a) mixing crystalline or semicrystalline Aliskiren with 50-150 wt. % of a brittle auxiliary and/or with at least 10 wt. % of a lubricant, in each case expressed in terms of the total amount of Aliskiren present, optionally with further auxiliaries;
(b) optionally compacting or dry granulating the mixture obtained in (a) and mixing the compacted or granulated Aliskiren mixture with one or more auxiliaries;
(c) dry pressing the mixture obtained in (a) or (b) into a tablet; and
(d) optionally coating the tablet obtained in (c).

22. A tablet obtained by the method according to claim 18.

23. A tablet containing Aliskiren, 20-70 wt. % of a brittle inorganic auxiliary, expressed in terms of the total weight of the tablet, and at least one disintegrant.

24. A tablet containing Aliskiren, at least 5 wt. % of a lubricant, expressed in terms of the total weight of the tablet, and at least one disintegrant.

25. The tablet according to claim 22, wherein said tablet is free from carbohydrates.

26. The tablet according to claim 22, wherein said tablet is characterised by an active agent release of at least 15% after 5 minutes, at least 40% after 10 minutes, at least 60% after 15 minutes, and at least 70% after 20 minutes, determined according to USP 28, Method <711>, Apparatus 2, in 500 ml 0.1 HCl pH 1.1 at 37° C. and 75 rpm.

27. The tablet according to claim 22, wherein said tablet has a hardness of at least 60 N, determined according to Pharm. Eur. 6.0, Method <2.9.8>.

28. A method for treating high blood pressure or an illness associated therewith, said method comprising the step of administering the pharmaceutical composition of claim 1, per se or in tablet form, to a patient in need thereof.

29. The method according to claim 28, wherein the illness associated with high blood pressure is selected from the group consisting of congestive heart failure, cardiohypertension, cardiofibrosis, postinfarct cardiomyopathy, complications as a consequence of diabetes, such as nephropathy, vasculopathy and neuropathy, coronary vascular diseases, restenosis after an angioplasty, increased intraocular pressure, glaucoma, abnormal vascular growth, hyperaldosteronism, states of anxiety and cognition disorders.

30. The method according to claim 28, wherein the pharmaceutical composition is administered in combination with a further active agent.

31. The method according to claim 30, wherein the further active agent is selected from the group consisting of AT₁ receptor antagonists, ACE inhibitors, beta blockers, calcium channel blockers, aldosterone synthase inhibitors, aldosterone receptor antagonists and diuretics.

32. The method according to claim 31, wherein the further active agent is hydrochlorothiazide.
